(19) United States
(12) Patent Application Publication
(10) Pub. No.: US 2005/0154291 A1
(43) Pub. Date: Jul. 14, 2005

(54) Method of using a small MRI scanner

(76) Inventors: Lei Zhao, Canton, MA (US); Grum Teklemariam, North Andover, MA (US); Jianyu Lian, Westford, MA (US)

Correspondence Address: Jacobs Patent Office, P.O. Box 390438, Cambridge, MA, 02139, US

(21) Appl. No.: 10/943,459

(22) Filed: Sep. 17, 2004

Related U.S. Application Data
(60) Provisional application No. 60/505,015, filed on Sep. 19, 2003.

(51) Int. Cl. A61B 5/05

(52) U.S. Cl. 600/410

(57) ABSTRACT

Multiple methods of performing whole body scans using a cost-effective small magnetic resonance imaging (MRI) system are disclosed. High magnetic field homogeneity of an open, small MRI is obtained by a combination of passive shimming and high order active shimming. A dynamic shimming while imaging (DSWI) method is provided to dynamically optimize field homogeneity for each scanned slab (slice) during imaging. Also provided is a method that scan a large subject volume only using a limited optimal imaging region of a magnet by continuously adjusting patient position and orientations with a 6 degrees of freedom patient table.

### PRIORITY CLAIM

This application claims priority from U.S. Provisional Application 60/505,015, filed Sep. 19, 2003.

### BACKGROUND OF THE INVENTION

Whole body imaging using magnetic resonance (MR) technology has been used for years. The cost of a whole body system is still extremely high, preventing it from being accessed worldwide, especially in rural settings, developing countries and emergency rooms of developed countries. These costs are driven by the major hardware that comprises an MRI system: the magnet, cryogenic systems, gradient coils, rf coils, patient table, the various amplifiers and image acquisition and processing subsystems. A whole body scanner typically requires a large enough magnet opening to accommodate whole body scans with sufficient magnetic field homogeneity, rf field homogeneity and enough rf power over large volumes to generate sufficient excitation, sufficient gradient linearity over a large volume, strength and slew rate to generate images of acceptable clarity and quality to make diagnosis of diseased organs and tissues. These in turn depend on the magnetic field strength and patient opening which determine to a large extent the overall system design, power consumption and demand on the complexity of the electronics and image acquisition and processing systems.

The magnet systems for MRI scanners have to accommodate the insertion of a human being and generate a homogeneous region large enough to cover a cylindrical area with a diameter between about 20 to about 50 cm, preferably about 40 cm, spherical volume (DSV) over the subject. For sufficient image quality, the magnets are typically made from permanent magnets in low-field systems (<5,000 gauss; <0.5 T) and superconducting magnet systems in high field systems (>10,000 gauss; >IT). To meet the demand of large fields and homogeneities of 10 parts per million (ppm) or less over the imaging region requires the presence of large magnets and magnet materials that makes the cost of the system very high. Similarly, the large imaging volumes also mean concomitant higher power consumptions by the gradient and rf systems as well. In superconducting magnet systems, this also translates into higher cryogenic fluid consumption which means higher maintenance and service costs.

In addition to the magnets, the gradient and rf magnetic subsystems require higher power. The gradients have to generate sufficiently strong gradient fields, with good linearity, that are switched fast over a large volume requiring higher voltages and currents. This makes the gradients and amplifier designs more complex and costly. Similarly, the rf coils have to provide sufficiently strong and homogeneous rf fields over large volumes. Once again the power required to achieve this scales strongly with volume and therefore the rf coils and amplifiers become more complex to design and costly as well. Furthermore, the higher power consumption requires special housing with heat ventilation for these amplifiers.

While magnetic resonance imaging (MRI) systems have to be large enough to be able to perform regular whole body MRI scans, this has been the root cause of the high system cost, preventing MRI from being used worldwide. Moreover, due to the large size of conventional MRI systems, interventional procedures are not easily implemented because physicians can not access the patients easily.

The present invention resolves these problems.

One solution to cutting MRI system costs is to reduce its size without reducing the patient bore size or patient gap. In a cylindrical horizontal MRI system, the bore length can be reduced. In an open, vertical MRI system, the magnet diameter and volume are reduced. For both approaches, however, the gradient coil and RF coil sizes will need to be reduced accordingly. The RF and gradient power consumption is then significantly reduced, putting less demand on the amplifiers. If an MRI system size is reduced to half its original size, the cost can be reduced by more than 35% of the original cost. However, reducing MRI system size has presented many problems which have caused image quality to deteriorate to such a great extent that small systems have not substantially penetrated the market.

A first consequence of size reduction is the shrinkage of the available imaging region. This means that most of the body may not be accessible for scanning in one session requiring multiple repositioning to scan all necessary regions and increasing scan time that otherwise would take less time in a conventional system. The reduction of magnet size also means a reduction in the gradient and rf sizes. Consequently, the gradients will have a less linear region causing more distortions closer to the center of the magnet than conventional systems. Likewise, the rf coils will have a less homogeneous region than conventional systems. Therefore, the overall field-of-view (FOV) is drastically reduced.

In prior art methods, when the target subject imaging area is larger than the available system FOV, a moving patient table is normally used to move the subject through the system FOV for scanning and thereby extend the available FOV. In these methods, the patient table pauses several times during a scan. When the patient table stops, images are acquired within the limited FOV. This move and pause action requires extra time and may shift the patient causing overlapping artifacts between different scanned images. For certain extreme cases when the table motion coincides with a certain encoding direction, the table can move continuously and phase errors generated can be compensated; however, this approach has restrictions on table motion and encoding directions, and can not be generally applied.

The present invention is directed to surmount all the above and similar problems.

### SUMMARY OF THE INVENTION

This invention is directed to methods of operating a whole body magnetic resonance imaging (MRI) systems that are much more cost-effective than current conventional MRI systems because of reducing the open magnet size and hence the size of RF and gradient coils. The size reduction for these key components results in a significant reduction in cost for magnet materials and for power supplies and amplifiers. Even though cost is lowered with a small scanner size, the imaging quality is preserved by the techniques of this invention. Due to the reduction in scanner size, the present system is more open than all conventional systems (both cylindrical and vertical open) and therefore more suitable for interventional MRI. The systems can either be permanent, electrical or superconducting based magnets.

A first requirement of the present invention is to reduce the magnet size significantly while keeping the whole body access and the magnetic field strength substantially the same as in conventional whole-body systems. For cylindrical horizontal magnet systems, the bore diameter can be kept the same while the bore length is made 20-30% shorter. For vertical open permanent magnet planar systems, the gap size is substantially maintained while the magnet diameter and total magnet volume are reduced by about 30 to 60% of a conventional system. This directly translates into a significant cutting of magnet material cost.

Along with the magnet size reduction, the gradient and RF coils are also reduced similarly in physical size. The smaller gradient or RF coils need much less power to achieve the same performance. As a result, the gradient and RF power amplifiers can be reduced significantly. Since the price of amplifiers is strongly related to the power required, the amplifier costs are also significantly reduced. Thus, the gradient and RF amplifier cost, another very expensive part of a MRI system, is also reduced, lowering the total system cost considerably.

A problem with simply reducing magnet, gradient coil and RF coil sizes is that it results in a reduction of optimal imaging area, i.e., the useful imaging area with sufficient field homogeneity, gradient linearity, and RF field homogeneity. Another problem is a decrease in field stability. A small scanner, especially a low field permanent magnet system, is more susceptible to temperature drifts than a large conventional scanner. This invention provides methods of dealing with these problems while benefiting from a significant cost reduction.

In accordance with this invention, a combination of passive shimming and high-order active shimming is utilized to improve magnetic field homogeneity in an open magnet to 10 ppm and less overall variation. During passive shimming, more attention is focused on avoiding the introduction of high order field inhomogeneity. Subsequently, multi-order active shims are used. The active shims are based on an orthonormal basis set, and more particularly they are based on a spherical harmonic set including zeroth, first, second, and higher order shimming coils which have an excellent ability to provide a further 100 ppm or more shimming capability. Since the shims are driven by currents, they are easily controlled. By integrating the shim controls into the MRI console an image based shimming method is developed for the best overall shimming possible.

In accordance with one aspect of this invention, the active shimming is combined with a “Dynamic Shimming while Imaging” (DSWI) technique to dramatically improve imaging plane field homogeneity where active shimming is optimal and the shimmed area is small. DSWI allows different imaging slices or slabs with different shimming coefficients within one scan to be shimmed individually. In this approach, after an MRI operator has chosen imaging slices or slabs, a different set of shimming parameters are acquired using field mapping pulse sequences for different imaging slices or slabs. These pre-acquired shimming parameter sets are stored in the MRI scanner and the actual imaging pulse sequence is applied. For each slice (or slab) excitation, encoding and data acquisition, the corresponding optimal shimming parameters are loaded before the play out of any gradient or RF pulses. The shimming parameter changes can be every TR (repetition time) or after multiple TRs, depending on encoding orders. Generally whenever slice position or orientation is changed, shimming coefficients are change accordingly. This procedure results in optimal field homogeneity for whole volume imaging.

To maintain thermal stability, an active thermal compensation system is used to stabilize magnet temperature and thus prevent field strength drifting. Due to the smaller amount of materials used in low field small permanent magnet systems as compared to conventional systems, the magnet temperature is more susceptible to ambient temperature changes which in turn affects the stability of the magnetic field strength. To overcome the temperature changes, thermal heating blocks with thermo-sensors are bound onto the magnet and its poles and an inverse feedback circuit is used to compensate for thermal changes at multiple locations. For better active thermal compensation the tolerances are made higher and more sensors and heaters used with faster response times.

With permanent magnets, temperature drift can change the center frequency during long imaging sessions. This problem is dealt with herein using a frequency monitor by directly and continuously sampling the center frequency during imaging and compensating any changes that arise. For example, during an imaging scan session, an additional RF coil with a NMR sensitive solution can be put into the magnet and the added RF coil can continuously (every TR or once a few TRs) acquire nuclear magnetic resonance signals when no gradients are turned on. The acquired signal is Fourier transformed and a new center frequency is extracted and adjusted so that the RF center frequency remains is the same as the magnetic field. Alternatively, the zeroth order active shim is used to adjust the magnet strength to maintain the original center frequency.

For a small MRI system, the sweet spots (i.e. the optimal imaging regions) are in the central part of the scanner. Also, gradient performance is better if the imaging plane (volume) is oriented so that it is always aligned with one of the inherent physical gradient directions (X, Y, Z) thereby avoiding large oblique planes. The desired subject imaging volume, however, can be larger than these regions, and their orientation can be arbitrary. The following methods developed in this invention solve this problem.

First, an automated patient table system with 6 degrees of freedom, is used so that the target subject imaging planes (volumes) are always manipulated to be align within the optimal regions. If a target imaging volume is larger than the optimal imaging region, the volume is divided into several slices (slabs), and each slice (slab) is aligned with the optimal imaging region. The patient table is automatically controlled to rotate around the magnet vertical center, tilt along its long and short axes, move up and down, move in and out of the scanner, and move from left to right (FIG. 2). This requires properly securing the subject on the patient table by either frames or straps to prevent the patient from moving or slipping. When imaging planes (volumes) are within the above defined imaging regions, field homogeneity and gradient and RF linearity are all optimized. Since imaging planes are aligned with the orthogonal magnet coordinates, logical gradients are aligned with the physical gradients. This approach improves gradient efficiency (high maximal gradient and fast gradient switching time) and lowers the requirement for the gradient amplifier power. Imaging at central parts of the magnet also reduces eddy current induction.

Second, a method is developed to obtain large subject volume images using a limited optimal imaging region. Imaging is always performed in the limited region, and objects move through the optimal region to obtain large coverage. This process is achieved by a “continuously no-pause moving table” and an adjustment of gradient fields to ensure the same gradient fields are seen by the subject during this motion. “Continuous movement” is used herein to mean a no-pause table movement, i.e. during the imaging of a plane or volume, the table continuously moves. In a normal image scanning setup, if an imaging plane (or volume) moves during excitation, encoding and acquisition, spins experience different gradients. This generates the wrong excitation, encoding, and acquisition information. In the present invention, the zeroth shimming is adjusted during a scan to compensate for the motion of the table. In this situation, a moving spin which moves along with the patient table will always experience the same gradient field as if it were stationary.

With a moving table scan, the imaging is always conducted in the optimal central region. When one plane (or volume) is scanned, a second plane (or volume) will roll into the optimal region for scanning. Therefore, as large an FOV as desired can be scanned. Because the table moves continuously, no time is wasted for inter-scan table pausing and starting delays. Also the move-and-pause approach of prior arts shifts patient positions during acceleration and deceleration. This problem is eliminated in the current continuous table movement procedure.

Third, the Dynamic Shimming while Imaging (DSWI) procedure may also be used in combination with the moving table as follows: before a scan, a patient table with a subject moves along the same path as the scan path. During the table movement, a few sample points are identified based on subject geometry. At a given sample point, the table stops and an optimal shimming coefficient set is determined using field mapping methods specific to the subject on the table at that sample point. Afterwards, all the shim coefficients are determined for all the sample points. The coefficients for each shimming channel are interpolated so that a continuous shimming coefficient set is obtained for different table (subject) positions. During image scanning, at the start of each TR, these sets of shimming coefficients are loaded so that the shim is always optimized at each sample point of the patient table as it moves through the imaging region.

An open whole body small scanner provides a large opening enabling the easy use of robotic controlled interventional devices for interventional MRI procedures. The low aspect ratio and high degree access of the design allows implementation of virtually unencumbered interventional techniques. For example, in an open vertical system, reducing the radius of the magnet while effectively keeping the same patient gap allows closer access to the patient. A typical scanner with a 40 cm DSV FOV will have a patient access space of 40 cm and magnet pole radius of 55 cm. Thus, reducing the DSV to 20 cm will reduce the radius of the magnet pole by 10 cm to 45 cm. Consequently, physicians can gain 10 cm more access to the patient. Also, its small fringe field allows for non-MRI compatible devices operated in the same scan room, enabling flexible clinical setup, especially for operating room setup. The replacement of MRI compatible devices with non-MRI compatible devices can significantly reduce clinical cost.

### BRIEF DESCRIPTION OF DRAWINGS

The features and advantages of the present invention will appear more clearly from the following description of a few non-limiting embodiments, illustrated in the attached drawings, in which:

FIG. 1 is a simplified block diagram showing the elements of an MRI system of this invention.

FIG. 2 shows the patient table orientations with 6 degrees of freedom movement. Thick line arrows show the 6 pairs of patient table moving directions.

FIG. 3 shows the optimal imaging planes oriented along the physical X, Y, and Z axes for a vertical permanent magnet MRI system;

FIG. 4 shows top views of the continuous table movement procedure during image scanning, beginning with (a) start of 1^{st} slab imaging; (b) end of 1^{st} slab imaging; and (c) end of whole volume imaging.

### DETAILED DESCRIPTION OF THE INVENTION

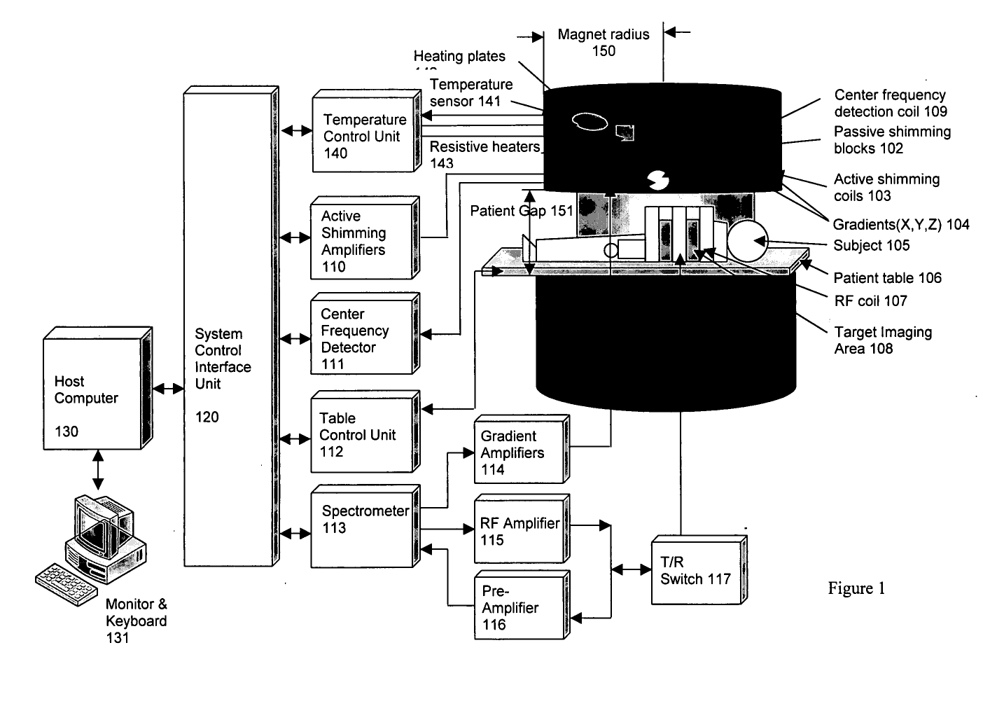

FIG. 1 illustrates a simplified block diagram of a MRI system for producing images in accordance with embodiments of the present invention. The system could have an open, cylindrical shaped electrical, superconducting, or a permanent magnet. An open, cylindrical permanent magnet is shown as an example in FIG. 1.

In the embodiment of this invention, the static magnet field is provided by the magnet 101 as shown in FIG. 1. In the preferred embodiment of this invention and as shown in FIG. 1 the field is oriented vertically; however, this invention applies to any other types of magnets and field orientation as well. Passive shimming blocks 102, mounted on the inner surfaces of the magnet, are used for improving the homogeneity of the magnetic field. These passive shimming blocks 102 are usually ferrite or high permeability materials. To further improve field homogeneity, active shimming coils 103 are also mounted within the gradient coils 104. The shimming coils generate, but are not limited to, 0^{th}, 1^{st}, 2^{nd} or higher order spherical harmonic gradient fields to compensate for the static field non-homogeneity. The current of the shimming coils are supplied by the active shimming amplifiers 110, which are controlled by the host computer 130 through the system control interface unit 120.

To prevent center frequency drifting due to temperature fluctuation, temperature sensors 141 are placed throughout the magnet 101. Based on the detected temperature changes, the temperature control unit 140 (including control circuits and amplifiers) will adjust the electrical current supplied to the heating plates 142 to stabilize the magnet temperature.

To further improve the stability of the system center frequency, a frequency stabilization system can be used. A small center frequency detection coil with a small phantom 109 can be placed in the magnet and be controlled by the center frequency detector 111. The detected center frequency will be used as feedback through the system control interface unit 120 to modulate the 0^{th} order shimming amplifier 110, so that the static magnetic field is stable.

To acquire images, a subject 105 lies on the patient table 106. A RF coil 107 is attached to the patient table (or mounted on the magnet after the gradient coil, typically referred to as a fixed RF body coil) that covers the subject's target imaging area 108. The RF coil excites and receives signals from the subject. These can be two separate RF coils, one for transmission and one for reception. Or it can be a single transmit/receive coil. In this case, a T/R switch 117 is needed to separate the transmitted and received signals. The RF signals generated by the spectrometer 113 are amplified by the RF amplifier 115 and then fed to the T/R switch 117. The received signal from the T/R switch 117 is sent to the spectrometer 113 through a pre-amplifier 116. The spatial selection and encoding is achieved by the X, Y, Z gradient fields generated by the gradient coils 103 mounted on the upper and lower magnet poles inside the patient gap. The gradient power is supplied by the gradient amplifiers 114, which are controlled by the spectrometer 113.

For a MRI scan, a pulse sequence is first designed based on the desired image contract. A pulse sequence composes of groups of RF and gradient pulses, and acquisition actions ordered at a certain time sequence. A pulse sequence is designed in the host computer 130 and downloaded to the spectrometer 113. During a scan, the spectrometer 113 generates gradient and RF pulses based the pulse sequence designed which are fed to the gradient amplifiers 114 and RF amplifiers 115, that then drive the gradient coils 103 and the RF coil 107 to generate the designed gradient and RF fields. After receiving the RF signals from the RF coil 107, the spins in the subject will emit certain RF signals with their spatial encoding from the gradient field generated by the gradient coils 103. The subject RF signals are first amplified by the pre-amplifier 116 after passing through a T/R switch 117. The amplified signal is then acquired by the spectrometer 113 and stored in the spectrometer as raw data. The raw data is finally sent to the host computer 130 via the system control interface unit 120. At the host computer, image reconstruction, primarily Fourier transform, is performed and subject images are displayed on the monitor 131.

An image pulse sequence normally requires a repetition of itself many times with the adjustment of a portion of the pulses. The duration of each repetition is called a TR (repetition time). The total scan time is the multiplication of TR and the total number of the repeats.

In the embodiment of this invention, the size of the magnet is significantly smaller, by up to 50% or more, than conventional magnets while preserving the magnetic field strength of a conventional magnet. More specifically, for a horizontal bore cylindrical magnet, the size reduction is in the length of the magnet. For an open, vertical field permanent magnet system, the size reduction is the magnet diameter and its total height. The diameter or length reduction can be as much as a quarter or higher. The total material volume reduced is even higher because the size varies quadratically with the radius.

Gradient coils 104 and fixed RF body coils in this embodiment are also reduced in size to fit into the small magnet. Because the power needed for gradient and RF coils is roughly proportional to the fourth and second power of the coil sizes, respectively, the required gradient and RF amplifiers in this embodiment have much smaller power requirements than conventional systems.

In this embodiment, we define the patient openness as the ratio of magnet radius 150 over patient gap 151. Due to the reduction in magnet radius 150, the openness in a circular magnet is improved from aspect ratios of 1.3 or greater to 1.1 or less.

To maintain thermal stability, the magnet is preheated using high power (>1 kW) resistors or plates 142 attached to the two poles of the magnet until a magnet temperature well above ambient is reached (>32° C.). Thereafter, smaller resistive heaters 143 (typically not greater than 50 W) uniformly distributed along the magnet exterior surfaces are used to heat the magnet. These heaters are controlled by a feedback mechanism where temperature sensors also uniformly distributed over the magnet to monitor the temperature throughout and the necessary amount of differential heat is applied to maintain a stable temperature of the magnet above ambient. Therefore, heat flows from the magnet to the environment and not from the environment to the magnet.

To ensure a stable magnetic field strength or center frequency, a frequency stabilization method is used, especially for a small permanent magnet scanner where center frequency drifting is a problem. In the embodiment of this invention, a small RF coil 109 with a small phantom is placed in the magnet. The center frequency detector 111, a single channel spectrometer, is used to drive the RF coil and acquire magnetic resonance signals. The excitation and acquisition of these signals takes place when the gradients are off. These signals are Fourier transformed and the center frequency is calculated in real-time. The center frequency offset is converted to a DC current which is then fed to the zeroth order active shimming coil 103. The field generated by the zeroth order shimming coil 103 is used to compensate the static magnetic field drifting.

In the embodiment of this invention, a combination of passive shimming using shimming blocks 102 and high-order active shimming 103 is used in an open magnet as shown in FIG. 1. Active shimming in the embodiment of this invention (consistent with the spherical harmonics) include 0^{th}, 1^{st} and 2^{nd} order shims (spanning the terms L=0, M=0; L=1, −L<M<+L; and L=2, −L<M<+L) for a total of 9 shims. Higher order shims, ones where the spherical harmonic terms are greater than or equal to L=3 and −L<M<+L are also used as needed, particularly, the m=0 with L=3, L=4, L=5 term shims, i.e. the axial orders.

During the passive shimming passes, the magnetic field in a region covering the imaging region is mapped and passive shimming blocks 102 are added first at the outer edges of the magnet pole face. The field is then remapped and based on the homogeneity changes either more elements are added or removed, and these steps are repeated several times until no further changes in the homogeneity are observed. Then, shim blocks are added at radii smaller than the previous one and the process of mapping the field, checking for homogeneity changes and adding or removing shim blocks at this smaller radius is repeated several times. When there are no further changes in the homogeneity, shim blocks get added at radii smaller than the previous ones. Then, the process of mapping the field, adding and/or removing shims, remapping the field and checking changes in the homogeneity, is repeated until no further changes are observed in the homogeneity. These steps are repeated until the center of the magnet pole face is reached. Once the center is reached, then starting at the outer edges of the magnet pole face shim blocks from the original placements are removed or added to check for any improvements in homogeneity. This check is done at all the radii that have shim blocks and the entire procedure is repeated several times until no improvements in field homogeneity over the region of interest, i.e., the imaging region is observed. This concludes the passive shimming passes.

Subsequently, active shimming can be performed. The advantage of active shimming in this setting is that the shims are based on an orthonormal basis and so each orthogonal component can be shimmed without much interaction with other terms. This is a significant advantage over the passive shimming passes and produces more than 100 ppm of shimming capability. The active shimming is done on a spherical phantom using image data. The phantom is filled with copper sulfate doped water. A phase sensitive pulse sequence is used to obtain a 3D phase map of the phantom. This phase map is subsequently converted to a magnetic field map. A least-mean-square fitting is conducted on the field map data to obtain the coefficients of the shims. Thereafter, using a scaling relation between shim current strength and these coefficients that has been pre-determined, the correction is applied.

To optimize the field homogeneity of the relevant imaging plane (volume), a Dynamic Shimming While Imaging (DSWI) method is applied. In this approach, a phase-sensitive pulse sequence is first applied and a 3D magnetic field strength map is obtained and stored in the host computer 130. During each imaging session, scan parameters, including slice (slab) locations, are first chosen by an operator. Right before image scanning starts, the active shimming coil 103 coefficients are calculated for each slice (slab) location of a multi-slice (-slab) scan (including one slice (slab) scan). Because the target shimming region for each slice (slab) is much smaller than a whole volume shimming approach, the field homogeneity is improved dramatically for each slice (slab). The calculated shimming coil coefficients are stored in an array with the same slice (slab) scan order of the imaging scan. During image scanning, right before each application of the RF or gradient pulses in a TR (repetition time), shimming amplifiers 110 are adjusted first to insure the corresponding imaging slice (slab) has the optimal field homogeneity. The adjustment of the shimming amplifiers 110 is achieved by retrieving the previously stored shimming coil coefficients triggered at the start of each TR.

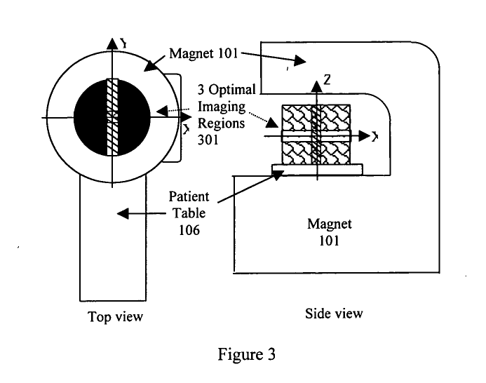

In the embodiment of this invention, three optimal imaging regions 301 are defined in FIG. 3. The optimal regions 301 are defined as those regions where field homogeneity, gradient linearity, RF field linearity and gradient performance are optimal. These regions are aligned with the X, Y, and Z axes typically covering 10 or more centimeters thickness. For a circular magnet 101, the optimal imaging regions are the three orthogonal slabs shown as the shaded regions 301 of FIG. 3. When imaging planes (volumes) are within these regions, logical gradients are aligned with physical gradients. Therefore, gradient performance (maximal gradient amplitude and slew rate) is optimal. In particular, when the need arises to perform oblique scans, the patient is oriented to the oblique angle rather than generating oblique gradients.

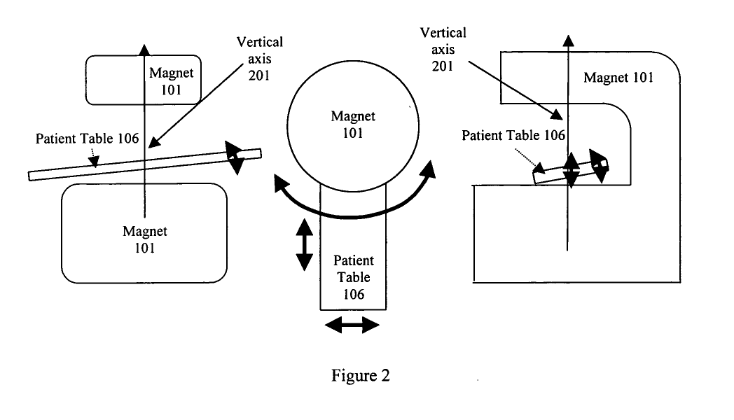

To further improve image quality, in another method of this invention, image scanning is limited to the optimal imaging regions 301. In this invention, a flexible table design is used to ensure this requirement is met in an open magnet configuration as shown in FIG. 2. This patient table can be oriented into a wide range of positions and orientations. The patient table is automatically controlled and has 6 degrees of movement, including moving left/right, up/down, in/out of the scanner, rotating around the scanner vertical axis 201 shown in FIG. 2, and tilt along the patient table's long and short axes. After an operator has defined imaging planes (volumes), a patient table moving path is calculated to ensure that imaging planes (volumes) pass through one of the optimal regions of the scanner. Image scanning is then synchronized with the movement of the patient table, so that scanning is only applied to planes (volumes) passing through the optimal imaging regions. To avoid patient shifting, frames or bands are used to safely secure the patient to the patient table.

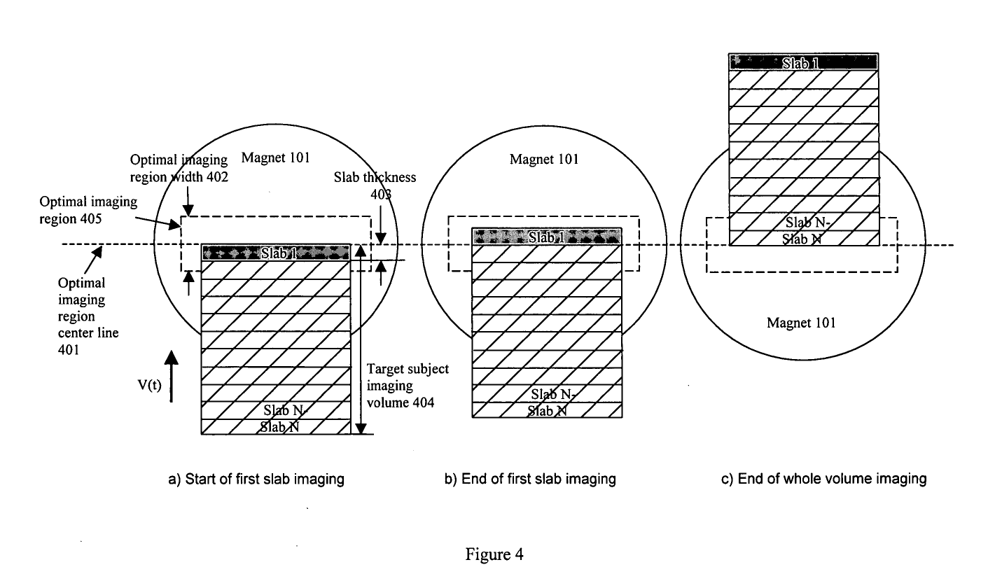

In this embodiment of this invention, the patient table 106 moves without any pause until the target subject imaging volume 404 has passed through the optimal imaging region 405, where image scanning is performed (see FIG. 4). To correct for the phase errors due to the table movement during excitation, encoding and acquisition, a magnetic field adjustment scheme is employed as explained below.

FIG. 4 shows the scanning procedure to image a large target imaging volume 404 in a limited optimal imaging region 405. First, the target imaging volume 404 is divided into N slabs (or slices). Then, the table position and orientation are adjusted by the table control unit 112 such that the imaging slabs are parallel to one of the optimal imaging regions 301. In the example of FIG. 4, the optimal imaging region is shown as 405. In this invention, the slab thickness 403 is smaller than half the width of the optimal imaging region 402. At the start of imaging, the leading edge of the target imaging volume 404 (also the leading edge of slab 1) is aligned with the center line of the optimal imaging region 401 (one of the central planar axes of the magnet) (see FIG. 4a). When the table starts moving at velocity V(t), the image scan starts. The direction of table motion is preferably orthogonal to 401. The speed is preferably constant and equal to 2*SLTH/T, where SLTH is the slab thickness and T is the total imaging time of a slab. With this speed, when a slab scan is finished, the trailing edge of the scanned slab aligns with the center of the optimal imaging region 401 (see FIG. 4b). The remaining slabs are imaged similarly with the same table velocity until the N^{th} slab is scanned.

In the above method, when the table is moving, the slab does not move through the same gradient fields. Therefore, yet in another invention, a method is developed to compensate for this problem. During image scan while the table is moving, the magnetic field strength is adjusted continuously by the zeroth order shimming coil 103. Consequently, if the combined gradient vector (physical X, Y and Z gradients) has a projection along the patient table moving direction, the magnetic field strength will be adjusted so that all moving spins experience the permanent static field plus a continuously varying zeroth order shimming field plus the gradient field with the net result being exactly the same as if the spins were stationary and they were experiencing just the permanent static field plus the gradient field. More specifically, if the amplitude of the combined imaging gradient vector projection along the patient table moving direction at time t (in units of second) is G(t) (in units of Tesla/meter), and the patient moving velocity is V(t) (in units of meter/second), then the zeroth order shimming field changing rate at time t is set to be the negative product, −G(t)*V(t). The minus sign reflects the fact that this is used to compensate for the field changes due to the motion of the patient table. When there is no gradient turned on (G(t)=0), the changing rate is zero.

After one slab is scanned, the zeroth order shimming coil current is reset back to its value before the start of this slab scan. Then, at the end of this first scan slab, the leading edge of the second slab is located at the central line of the optimal imaging region 401 as shown in FIG. 4b. As the table continues to move, the scanning as in the first slab scan continues. If the slab sections have gaps, the table will move until the leading edge of the second slab aligns with 401 before the scanning starts. Using this method, there is no change in the pulse sequence itself, except that the patient table motion is synchronized with the slab scanning compensation scheme described above.

Before image scanning starts, the zeroth order shimming values for the entire scan are pre-calculated based on the designed gradient projection along the direction of the patient table motion and the velocity of the table motion using −G(t)*V(t). The values are stored in the system control interface unit 120 or the host computer 130. There are several methods to store and retrieve the zeroth order shim compensation values.

In the first method, one can store the zeroth order shimming values for the whole scan (all slabs) at increments of the minimal gradient dwell time, e.g., 1 microsecond per step (increment). Then, the shimming values are continuously fed to the zeroth order shimming amplifier channel 103 starting at the trigger of the onset of the entire image scan. To avoid issues of synchronization between the shimming value changes and the imaging gradient changes, gradient variations can be grouped in many stages over the course of the entire scan and a separate series of start and stop triggers used to maintain the synchronization between the gradient variations and the shim amplifier 103. For each group, a group of continuous zeroth order shimming values are calculated and stored in the system control interface unit 120 or the host computer 130. During image scanning, at all gradient starting points a trigger is generated by the spectrometer 113 programmed in the imaging pulse sequence. This trigger triggers the system control interface unit or the host computer to play out the corresponding group of zeroth order shimming values. To summarize, using these methods, a large FOV as desired can be imaged as long the fixed RF body coil is utilized.

When using the above zeroth order shimming adjustment method, the frequency-stabilization method described earlier will use the pre-calculated adjusted magnetic field values as reference instead of keeping it constant.

To obtain optimal shimming, the Dynamic Shimming While Imaging (DSWI) method is applied to scan in the optimal imaging region. When a subject passes through the optimal imaging region, the field homogeneity can vary due to the susceptibility effects generated by the interaction of the different parts of the subject with the central part of the magnetic field. So, shimming is performed dynamically during the table motion as follows. After the scan path is planned, the patient table will move along the same path as the imaging path. During the table movement, the table stops at a select predefined sample points. The sample points are picked according the geometric variation of the subject. If the subject exhibits minimal geometric variations, fewer sample points are needed. If the subject exhibits many geometric variations more sample points are preferred. For example, for a head scan where the table can move through 30 cms, the number of sample points can be 4-5. At a sample point, the field homogeneity of the optimal imaging region is acquired using field-mapping sequences, such as a gradient echo sequence with different echo times. The field map is used to calculate the shimming coefficients of the targeted optimal imaging region. The coefficients are then converted to shimming coil current values as described previously. The coefficients are for all shimming coils. For this embodiment, it corresponds to 0^{th}, 1^{st} and 2^{nd} order shim fields. After the fields for all the sample points have been mapped, each shimming coil has several coefficients corresponding to different sample points of different table positions. The coefficients of each shim coil at the sample points are interpolated to obtain continuous shimming values. During imaging, as the patient table moves, the corresponding shimming values are loaded to maintain optimal field homogeneity at the optimal target region. Because the target shimming region is restricted to a limited region at the central part of the magnet, the shimming result is optimal.

1. A whole body MRI system design with a reduced volume that significantly reduces the material costs of magnet, gradient and RF coils.

2. A whole body MRI system design with reduced gradient and RF coil size which significantly lowers gradient and RF power consumption and therefore lowered amplifier and operation costs.

3. A small MRI design that has large patient openness with an aspect ratio of 1.1 or less.

4. A method that uses thermal heaters and a feedback mechanism to stabilize temperature drifts of a small permanent magnet.

5. A method that uses a detection coil and a zeroth order shimming coil to detect and compensate for static field or center frequency drifts on a small MRI system.

6. A design that uses a combination of passive shimming and high order active shimming to obtain high field homogeneity in an open MRI system,
(a) wherein passive shimming is initially performed using shimming blocks placed at the outer radii of the magnet pole edge until the homogeneity changes from field maps over the imaging region of interest is no longer improved;
then this process repeated to a next inner radii and so on until the center is reached and the entire process repeated starting again from the outer radii and checking for homogeneity improvements by adding or removing blocks and working again towards the center;
(b) the passive shimming is iterated until no further improvements are achieved in homogeneity over the imaging region of interest;
(c) wherein active shims generate fields based on the spherical harmonic basis set, and shim values obtained from a 3D field map using a phase sensitive imaging pulse sequence are then used to obtain up to a further 100 ppm or more capability in shimming.

7. A method that uses active shimming coils to dynamically shim individual slices (slab) of a scanned volume separately during an image scan,
(a) wherein a 3D field map is initially acquired,
(b) said map is used to calculate shim coefficients,
(c) said sets of shimming coefficients are calculated for each slice (slab) prior to imaging, and
(d) during imaging, the set of shimming coefficients corresponding to the slice (slab) to be scanned are loaded at the trigger of the start of each TR to generate the optimal field homogeneity for the particular slice (slab).

8. An imaging method that uses only limited optimal imaging regions for whole body imaging,
(a) wherein oblique planes are imaged by adjusting the patient orientation (with the help of, but not limited to, 6 degrees of movement of a patient table), rather than by turning on multiple gradients at the same time,
(b) wherein the target imaging volume is divided into several slabs (slices), whose thickness is equal or less than half the optimal imaging region width. Each slab is scanned in the optimal imaging region during the table movement. The patient table continuously moves during imaging until the whole target imaging volume has passed through the optimal imaging region,
(c) wherein during imaging, the zeroth order shimming value changes are synchronized with the table movement to compensate for the gradient changes from the table motion.

9. A method that uses active shimming coils to dynamically shim the same optimal imaging region with different subject volume during a moving table scan,
(a) wherein before imaging, a subject on the patient table moving along the same imaging path stops at several sample points and a field map is acquired by a field-mapping pulse sequence to obtain optimal active shimming coefficient sets for the sample point and then interpolated to generate a continuous shim,
(b) wherein during imaging scan, stored optimal shimming coefficient sets are retrieved and optimally shim the magnetic field.

10. A system with low aspect ratio and high degree access allows implementation of virtually unencumbered interventional techniques.
